(19) United States
(12) Patent Application Publication
(10) Pub. No.: US 2007/0191467 A1
(43) Pub. Date: Aug. 16, 2007

(54) STATIN AND OMEGA-3 FATTY ACIDS FOR LIPID THERAPY

(75) Inventors: Roelof M.L. Rongen, Califon, NJ (US); George Bobotas, Tarpon Springs, FL (US); Egil Bodd, Oslo (NO); Hogne Vik, Eiksmarka (NO)

Correspondence Address: ARENT FOX PLLC, 1050 CONNECTICUT AVENUE, N.W., SUITE 400, WASHINGTON, DC, 20036, US

(73) Assignee: RELIANT PHARMACEUTICAL, INC., LIBERTY CORNER, NJ (US)

(21) Appl. No.: 11/742,292

(22) Filed: Apr. 30, 2007

Related U.S. Application Data
(63) Continuation-in-part of application No. 11/284,095, filed on Nov. 22, 2005.
(60) Provisional application No. 60/633,125, filed on Dec. 6, 2004.
(60) Provisional application No. 60/659,099, filed on Mar. 8, 2005.
(60) Provisional application No. 60/699,866, filed on Jul. 18, 2005.
(60) Provisional application No. 60/840,012, filed on Aug. 25, 2006.
(60) Provisional application No. 60/850,280, filed on Oct. 10, 2006.
(60) Provisional application No. 60/852,398, filed on Oct. 18, 2006.

(51) Int. Cl. A61K 31/401; A61K 31/366; A61K 31/22; A61K 31/202

(52) U.S. Cl. 514/423; 514/460; 514/548; 514/560

(57) ABSTRACT

A method of lipid therapy, comprising providing a subject group having a baseline triglyceride level of 200 to 499 mg/dl and being at or near its low-density lipoprotein cholesterol (LDL-C) level goal, and reducing the triglyceride level and the non-high-density lipoprotein cholesterol (non-HDL-C) level of the subject group as compared to treatment with a 3-hydroxy-3-methyl glutaryl coenzyme A (HMG CoA) inhibitor alone, by administering to the subject group an effective amount of an HMG CoA inhibitor and a composition comprising omega-3 fatty acids.

The present application is a continuation-in-part of application Ser. No. 11/284,095, filed Nov. 22, 2005, which claims priority from provisional patent application Ser. No. 60/633,125, filed Dec. 6, 2004, Ser. No. 60/659,099, filed Mar. 8, 2005, and Ser. No. 60/699,866, filed Jul. 18, 2005. The present application also claims priority from provisional patent application Ser. No. 60/840,012, filed Aug. 25, 2006, Ser. No. 60/850,280, filed Oct. 10, 2006, and Ser. No. 60/852,398, filed Oct. 18, 2006. The disclosure of the parent and priority applications is hereby incorporated by reference.

### FIELD OF THE INVENTION

The present invention relates to a method utilizing a single administration or a unit dosage of a combination of an HMG-CoA inhibitor and omega-3 fatty acids for the treatment of patients with hypertriglyceridemia or hypercholesterolemia or mixed dyslipidemia, coronary heart disease (CHD), vascular disease, atherosclerotic disease and related conditions, and for the prevention or reduction of cardiovascular, cardiac, and vascular events.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (TC), LDL-C, and apolipoprotein B (Apo-B, a membrane complex for LDL-C and VLDL-C) promote human atherosclerosis and decreased levels of HDL-C and its transport complex, apolipoprotein A (Apo-A), which are associated with the development of atherosclerosis. Further, cardiovascular morbidity and mortality in humans can vary directly with the level of TC and LDL-C and inversely with the level of HDL-C. In addition, researchers have found that non-HDL cholesterol (non-HDL-C) is an important indicator of hypertriglyceridemia, vascular disease, artherosclerotic disease and related conditions.

Cardiovascular disease (CVD) is a broad term that encompasses a variety of diseases and conditions. It refers to any disorder in any of the various parts of the cardiovascular system, which consists of the heart and all of the blood vessels found throughout the body. Diseases of the heart may include coronary artery disease, CHD, cardiomyopathy, valvular heart disease, pericardial disease, congenital heart disease (e.g., coarctation, atrial or ventricular septal defects), and heart failure. Diseases of the blood vessels may include arteriosclerosis, atherosclerosis, hypertension, stroke, vascular dementia, aneurysm, peripheral arterial disease, intermittent claudication, vasculitis, venous incompetence, venous thrombosis, varicose veins, and lymphedema. Some patients may have received treatment for their CVD, such as vascular or coronary revascularizations (angioplasty with or without stent placement, or vascular grafting). Some types of cardiovascular disease are congenital, but many are acquired later in life and are attributable to unhealthy habits, such as a sedentary lifestyle and smoking. Some types of CVD can also lead to further heart problems, such as angina, major adverse cardiovascular events (MACEs) and/or major coronary events (MCEs) such as myocardial infarction (MI) or coronary intervention, or even death (cardiac or cardiovascular), which underscores the importance of efforts to treat and prevent CVD.

Primary prevention efforts are focused on reducing known risk factors for CVD, or preventing their development, with the aim of delaying or preventing the onset of CVD, MACEs or MCEs. Secondary prevention efforts are focused on reducing recurrent CVD and decreasing mortality, MACEs or MCEs in patients with established CVD.

MACEs include cardiac death, other cardiovascular death, MCEs (which include myocardial infarction (Ml) and coronary intervention such as coronary revascularization, angioplasty, percutaneous transluminal coronary angioplasty (PTCA), percutaneous coronary intervention (PCI) and coronary artery bypass graft (CABG)), hospitalization for unstable angina, stroke, transient ischemic attack (TIA) and hospitalization for peripheral artery disease (PAD).

The Third Report of the National Cholesterol Education Program Expert Panel on Detection, Evaluation, and Treatment of High Blood Cholesterol in Adults, NIH Publication No. 02-5215 (September 2002) (also known as the “NCEP ATP III”), hereby incorporated by reference, provides recommendations for cholesterol-lowering therapy in an effort to reduce risk of CHD. In the ATP III, CHD is defined as symptomatic ischemic heart disease, including Ml, stable or unstable angina, demonstrated myocardial ischemia by noninvasive testing, and history of coronary artery procedures. The ATP III indicates that LDL-C is the primary target of lipid therapy, with other lipids to be controlled including triglycerides (TG), non-HDL-C and HDL-C.

A guiding principle of ATP III is that the intensity of LDL-C lowering therapy is adjusted to the individual's absolute risk for CHD. Risk assessment is broken down into short term (≦10-year) and long term (>10-year) risk of CHD, and the LDL-C goals are adjusted accordingly. In addition, ATP III identifies three categories of risk for CHD that modify LDL-C goals: established CHD and CHD risk equivalents, multiple (2+) risk factors, and 0-1 risk factor. Established CHD and CHD risk equivalents include CHD, other clinical atherosclerotic diseases, diabetes mellitus, and multiple risk factors and a 10-year risk for CHD >20 percent. The major independent risk factors identified in risk factor counting include cigarette smoking, hypertension, low HDL-C, family history of premature CHD and age.

The LDL-C goals for the three categories of risk factors are as follows:

| Risk Factors | LDL-C Goal |
|---|---|
| CHD and CHD Risk Equivalent | <100 mg/dl |
| Multiple (2+) Risk Factors | <130 mg/dl* |
| 0-1 Risk Factor | <160 mg/dl |

*LDL-C goal for multiple risk factor persons with 10-year risk >20 percent is <100 mg/dl.

The ATP III also outlines LDL-C goals for patients based on the percentage of 10-year risk for CHD:

| 10-Year Risk | LDL-C Goal |
|---|---|
| >20% | <100 mg/dl |
| 10-20% | <130 mg/dl |
| <10% and Multiple (2+) Risk Factors | <130 mg/dl |
| <10% and 0-1 Risk Factor | <160 mg/dl |

3-hydroxy-3-methyl glutaryl coenzyme A (HMG-CoA) reductase inhibitors (known as HMG-CoA inhibitors, or “statins”), have been used to treat hyperlipidemia and atherosclerosis, for example. Typically, statin monotherapy has been used to treat cholesterol levels, particularly when a patient is not at an acceptable LDL-C level. Statins inhibit the enzyme HMG-CoA reductase, which controls the rate of cholesterol production in the body. Statins lower cholesterol by slowing down the production of cholesterol and by increasing the liver's ability to remove the LDL-C already in the blood. Accordingly, the major effect of the statins is to lower LDL-C levels. Statins have been shown to decrease CHD risk by about one-third. However, statins only appear to have a modest effect on the TG-HDL axis.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII phospholipid complex. Further, omega-3 fatty acids seem to be well tolerated, without giving rise to any severe side effects.

One such form of omega-3 fatty acids is a concentrate of omega-3, long chain, polyunsaturated fatty acids from fish oil containing DHA and EPA and is sold under the trademark Omacor®. Such a form of omega-3 fatty acids is described, for example, in U.S. Pat. Nos. 5,502,077, 5,656,667 and 5,698,594, each incorporated herein by reference.

In many patients with hypertriglyceridemia, hypercholesterolemia or mixed dyslipidemia, the use of diet and single-drug therapy does not always decrease LDL-C and TG adequately enough to reach targeted values. In these patients, a complementary combination therapy of a statin and omega-3 fatty acids may be desirable.

Many studies have examined the effects of fish oil and statin therapy.

Nakamura et al. investigated the effects of purified EPA and statins on patients with hyperlipidemia. Patients having baseline triglyceride levels of 2.07 mmol/l (about 182 mg/dl) and already treated with 5-20 mg/day pravastatin or 5 mg/day simvastatin were additionally treated for 3 months with 900 or 1800 mg/day purified (>90%) EPA ethyl ester. It was reported that combination treatment significantly reduced triglyceride levels, and significantly increased HDL-C levels, as compared to baseline monotherapy. LDL-C levels were not reported. Nakamura et al., Int. J. Clin. Lab Res. 29:22-25 (1999).

Davidson et al. investigated the effects of marine oil and simvastatin in patients with combined hyperlipidemia. Patients having baseline triglyceride levels of 274.7 mg/dl to 336.8 mg/dl, and baseline LDL-C levels of 160 mg/di to 240 mg/dl, were treated for 12 weeks with 10 mg/day simvastatin and placebo, 7.2 g/day marine oil (SuperEPA® 1200) and placebo, or a combination of simvastatin and SuperEPA®. The content of omega-3 fatty acids in 7.2 g of marine oil used in the study was 3.6 g, with an EPA/DHA ratio of 1.5. Combination treatment was shown to significantly increase HDL-C levels, as compared to marine oil alone. In addition, triglyceride and non-HDL-C levels were significantly reduced with combination treatment, as compared to baseline. Davidson et al., Am. J. Cardiol. (1997) 80: 797-798.

Hong et al. investigated the effects of fish oil and simvastatin in patients with coronary heart disease and mixed dyslipidemia. Patients having baseline triglyceride levels of 292.8 mg/dl or 269.5 mg/dl, and baseline LDL-C levels of 89.4 mg/dl or 89.6 mg/dl, were initially treated with 10-20 mg/day simvastatin for 6-12 weeks. Thereafter the patients were treated with simvastatin and placebo or simvastatin and 3 g/day fish oil (Meilekang™). Combined treatment significantly reduced triglyceride levels, as compared to baseline and placebo. In addition, combined treatment numerically increased HDL-C levels, and numerically reduced LDL-C levels, as compared to baseline. However, the changes in HDL-C levels and LDL-C levels were not statistically significant. Hong et al., Chin. Med. Sci. J. 19:145-49 (2004).

Contacos et al. investigated the effects of fish oil and pravastatin on patients with mixed hyperlipidemia. Patients having baseline triglyceride levels of 4.6 to 5.5 mmol/l (about 404 to 483 mg/dl) and baseline LDL-C levels of 4.4 to 4.9 mmol/I (about 170 to 189 mg/dl) were initially treated for 6 weeks with 40 mg/day pravastatin, 6 g/day fish oil (Himega™, containing 3 g of omega-3 fatty acids, with an EPA/DHA ratio of 2:1), or placebo. Thereafter, all patients were treated with pravastatin and fish oil for an additional 12 weeks. Initial treatment with pravastatin significantly reduced LDL-C levels. Combined treatment of pravastatin and fish oil also significantly reduced triglyceride and LDL-C levels. However, the addition of fish oil to pravastatin monotherapy resulted in only a numerical increase in LDL-C levels, which was not statistically significant. Treatment with fish oil alone significantly reduced triglyceride levels, but increased LDL-C levels. Combined treatment for this group significantly reduced LDL-C levels, as compared to fish oil alone (but not as compared to baseline). Contacos et al., Arterioscl. Thromb. 13:1755-62 (1993).

Singer investigated the effects of fish oil and fluvastatin on patients with combined hyperlipidemia. Patients having baseline triglyceride levels of 258 mg/dl and baseline LDL-C levels of 178 mg/dl were initially treated for two months with 40 mg/day fluvastatin, and thereafter were additionally treated for two months with 3 g/day fish oil (18% EPA and 12% DHA). Thereafter, the patients remained on fluvastatin therapy alone for a final two months. Fluvastatin monotherapy was shown to significantly reduce triglyceride and LDL-C levels, and significantly increase HDL-C levels. Combination treatment also significantly reduced triglyceride and LDL-C levels. Singer, Prost. Leukotr. Ess. Fatty Acids 72:379-80 (2005).

Liu et al. investigated the effects of fish oil and simvastatin in patients with hyperlipidemia. Patients having baseline triglyceride levels of 1.54 to 1.75 mmol/l (about 136 to 154 mg/dl), and baseline LDL-C levels of 4.46 to 4.67 mmol/l (about 172 to 180 mg/dl), were treated for 12 weeks with 10 mg/day simvastatin, 9.2 g/day fish oil (Eskimo-3), or a combination of simvastatin and Eskimo-3. The fish oil contained 18% EPA, 12% DHA, and a total of 38% omega-3 fatty acids. Combined treatment significantly reduced triglyceride and LDL-C levels, and significantly increased HDL-C levels, as compared to baseline, and significantly reduced triglyceride levels as compared to simvastatin alone. Liu et al., Nutrition Research 23 (2003) 1027-1034.

Grekas et al. examined the combined treatment of low-dose pravastatin and fish oil (prolipid, 1 g daily) in post-renal transplantation dislipidemia patients having baseline triglyceride levels of 169 mg/dl or 184 mg/dl, and baseline LDL-C levels of 151 mg/dl or 163 mg/dl. Pravastatin treatment significantly reduced LDL-C as compared to baseline. The combination of pravastatin and fish oil significantly reduced triglycerides and LDL-C as compared to baseline. Grekas et al., Nephron (2001) 88: 329-333.

Howe et al. treated patients with persistent mild hypertriglyceridemia and already on statin therapy with 4 g/day or 8 g/day tuna oil (HiDHA™) or placebo for 6 months. Patients had baseline triglyceride levels of 2.2 mmol/l (about 200 mg/dl). Triglycerides and non-HDL-C levels were significantly reduced in patients administered 8 g/day of fish oil. LDL-C levels were raised with fish oil treatment, although the change was not significant. There was no significant change in HDL-C levels. Howe et al., Clin. Exp. Pharmacol. 29: A50-A51 (2002).

Sandset et al. examined the influence of combination treatment with simvastatin (40 mg/day) and omega-3 fatty acids (4 g/day Eicomarine 60, containing 28% EPA, 36% DHA and 6% DPA) on patients with hypercholesterolemia. Patients having baseline triglyceride levels of 1.12 mmol/l (about 102 mg/dl) and baseline LDL-C levels of 7.40 mmol/l (about 286 mg/dl) had a significant reduction in triglycerides and LDL-C levels with statin monotherapy, compared to baseline, but only a significant reduction in LDL-C with combination treatment, as compared to baseline. Sandset et al, Arterioscler. Thromb. Vasc. Biol. 11:138-45 (1991).

Tomei et al. evaluated the effects of administration of a combination of simvastatin (10 mg/day) and omega-3 fatty acids (2 g/day of concentrated omega-3 fatty acids containing not less than 85% EPA and DHA) on subjects afflicted with coronary artery disease, hypercholesterolemia and moderate hypertriglyceridemia. Subjects having baseline triglyceride levels of 3.37 mmol/l (about 306 mg/dl) or 3.47 mmol/l (about 315 mg/dl), and baseline LDL-C levels of 4.59 mmol/l (about 177 mg/dl) or 4.82 mmol/l (about 186 mg/dl) showed a significant reduction in triglycerides and LDL-C levels with both simvastatin monotherapy and combination therapy, as compared to baseline. Only with respect to triglycerides was there a significant difference between monotherapy and combination therapy. Tomei et al., Cardiologia, 38: 773-78 (1993).

Nordoy et al. examined the effects of administration of a combination of lovastatin and K-85 (ethyl ester of EPA and DHA containing 95% n-3 fatty acids) to patients with familial hypercholesterolemia having triglyceride levels of 1.5 mmol/l (about 137 mg/dl) or 1.7 mmol/l (about 155 mg/dl) and LDL-C levels of 7.1 to 7.5 mmol/l (about 274 to 290 mg/dl). Table 1 discloses the effect of K-85 alone, lovastatin alone, and the combination of K-85 and lovastatin on total cholesterol, triglycerides, HDL-cholesterol and LDL-cholesterol. Lovastatin treatment significantly decreased triglycerides and LDL-C, and significantly increased HDL-C, as compared to baseline. The combination of K-85 and lovastatin significantly decreased triglycerides and LDL-C as compared to baseline. Nordoy et al., Essent. Fatty Acids Eicosanoids, Invited Pap. Int'l Congr. 3^{rd}, 252-56 (1992).

Studies have also investigated the effect of statins and Omacor® omega-3 fatty acids.

Hansen et al. investigated the effect of lovastatin (40 mg/day) in combination with fish oil concentrate (6 g/day Omacor® omega-3 fatty acids) in patients with hypercholesterolemia. Patients having baseline triglyceride levels of 1.66 mmol/l (about 146 mg/dl) and baseline LDL-C levels of 7.69 mmol/l (about 290 mg/dl) were treated with 6 g/day Omacor® for 6 weeks, followed by 40 mg/day lovastatin for an additional 6 weeks, and a combination of both Omacor® and lovastatin for a final 6 weeks. Lovastatin monotherapy resulted in significant increases in HDL-C levels, and significant decreases in triglyceride and LDL-C levels, compared to baseline. After combination treatment, triglyceride and LDL-C levels were further decreased, with such decrease being deemed significant as compared to baseline. Hansen et al., Arteriosclerosis and Thrombosis 14(2): 223-229 (February 1994).

Nordoy et al. investigated the effect of atorvastatin and omega-3 fatty acids on patients with hyperlipemia. Patients having baseline triglyceride levels of 3.84 mmol/l (about 337 mg/dl) or 4.22 mmol/l (about 371 mg/dl), and baseline LDL-C levels of 4.89 mmol/l (about 189 mg/dl) or 5.31 mmol/l (about 205 mg/dl) were treated with 10 mg/day atorvastatin for 5 weeks. Thereafter, for an additional 5 weeks, atorvastatin treatment was supplemented with 2 g/day Omacor® or placebo. Atorvastatin monotherapy significantly increased HDL-C levels, and triglyceride and LDL-C levels significantly decreased, as compared to baseline. Combination treatment further increased HDL-C levels, as compared to atorvastatin alone. The effects attributable to the omega-3 fatty acids were not significant with respect to triglyceride and LDL-C levels. Nordoy et al., Nutr. Metab. Cardiovasc. Dis. (2001) 11:7-16.

Salvi et al. investigated the effects of Omacor® and simvastatin on patients with familial hypercholesterolemia. Patients having baseline triglyceride levels of 1.355 mmol/l (about 119 mg/dl) and baseline LDL-C levels of 5.52 mmol/l (about 213 mg/dl), and already treated with 20-40 mg/day simvastatin were additionally treated with 6 g/day Omacor® for 4 weeks. It was shown that combination treatment significantly decreased triglyceride and LDL-C levels after 2 weeks, as compared to baseline monotherapy. Salvi et al., Curr. Ther. Res. 53:717-21 (1993).

Bhatnagar et al. investigated the effects of omega-3 fatty acids (2 g Omacor® omega-3 fatty acids twice a day) for treating subjects with established CHD and type IIb hyperlipidemia who were already taking simvastatin. The subjects had baseline triglyceride levels of 3.8 mmol/l or 4.6 mmol/l (about 345 mg/dl to 418 mg/dl), and baseline LDL-C levels of 3.5 mmol/l or 4.2 mmol/l (about 135 mg/dl or 162 mg/dl). Combination treatment significantly lowered triglyceride levels as compared to baseline. Bhatnagar et al., Eur. Heart J Supplements (2001) 4 (Suppl. D): D53-D58.

Chan et al. studied the combined treatment of atorvastatin (40 mg/day) and fish oil (4 Omacor® omega-3 fatty acid capsules orally at night, 4 g/day) on obese, insulin-resistant men with dyslipidemia studied in a fasted state. Patients having baseline triglyceride levels of 1.7 to 2.0 mmol/l (about 150 to 170 mg/dl), and baseline LDL-C levels of 3.80 to 3.92 mmol/l (about 147 to 151 mg/dl), were treated for 6 weeks with: 40 mg/day atorvastatin and placebo; 4 g/day Omacor® and placebo; a combination of atorvastatin and Omacor®; or a combination of placebos. With combination treatment, the main effects from the fish oil were significant with respect to triglycerides and HDL-C levels, but the main effects from the fish oil were not significant with respect to non-HDL-C and LDL-C. Chan et al., Diabetes, 51: 2377-2386 (August 2002).

Chan et al. also investigated the effects of atorvastatin (40 mg/day) and fish oil (4 g/day Omacor® omega-3 fatty acids at night) on obese men with dyslipidemia and insulin resistance, having baseline triglyceride levels of 1.9 to 3.0 mmol/l (about 173 to 273 mg/dl), and baseline LDL-C levels of 3.80 to 3.92 mmol/l (about 147 to 151 mg/dl). The treatment groups received a placebo, atorvastatin, Omacor® omega-3 fatty acids, or a combination thereof at night. With combination treatment, the main effects from the fish oil were significant with respect to triglycerides and HDL-C levels, but the main effects from the fish oil were not significant with respect to non-HDL-C and LDL-C. Chan et al., Eur. J. of Clin. Invest. (2002) 32: 429-436.

Nordoy et al., investigated the effect of omega-3 fatty acids (4 g/day Omacor® omega-3 fatty acids) and simvastatin (20 mg/day) on patients with hyperlipidemia having baseline triglyceride levels of 4.42 mmol/l (about 402 mg/dl) or 5.08 mmol/l (about 462 mg/dl). Baseline LDL-C levels were not reported. Treatment with simvastatin alone decreased LDL-C and increased HDL-C. After the addition of omega-3 fatty acid supplementation, a significant reduction in triglycerides was noted, as compared to placebo, without a significant increase in LDL-C or HDL-C. Essent. Fatty Acids Eicosanoids, Invited Pap. Int'l Congr. 4^{th}, 256-61 (1998).

Nordoy et al., also investigated the efficiency and the safety of treatment with simvastatin and omega-3 fatty acids in patients with hyperlipidemia. Patients having baseline triglyceride levels of 2.76 mmol/l (about 243 mg/dl) or 3.03 mmol/l (about 266 mg/dl), and baseline LDL-C levels of 3.90 mmol/l (about 151 mg/dl) or 4.43 mmol/l (about 171 mg/dl), were treated for 5 weeks with 20 mg/day simvastatin or placebo, then all patients were treated for an additional 5 weeks with 20 mg/day simvastatin. Thereafter, patients were additionally treated with 4 g/day Omacor® or placebo, for a further 5 weeks. With combination treatment, the main effects from the fish oil were significant with respect to triglyceride levels, but the main effects from the fish oil were not significant with respect to HDL-C and LDL-C levels. Nordoy et al., J. of Internal Medicine, 243:163-170 (1998).

Durrington et al. examined the effectiveness, safety, and tolerability of a combination of Omacor® omega-3 fatty acids and simvastatin in patients with established coronary heart disease and persisting hypertriglyceridemia. Patients having baseline triglyceride levels of 3.8 mmol/l (about 345 mg/dl) or 4.6 mmol/l (about 418 mg/dl), and baseline LDL-C levels of 3.5 mmol/l (about 135 mg/dl) or 4.2 mmol/l (about 162 mg/dl) were treated with 10-40 mg/day simvastatin and 2 g/day Omacor® or placebo, for 24 weeks in a double-blind trial, after which both groups were invited to receive Omacor® for a further 24 weeks in an open study. Combination treatment significantly decreased triglyceride levels within 12 weeks, as compared to baseline monotherapy. LDL-C levels significantly decreased, as compared to baseline monotherapy, after 48 weeks. Durrington et al., Heart, 85:544-548 (2001).

U.S. Patent Application Publication No. 2003/0170643 claims a method of treating a patient, by administering a therapeutic which lowers plasma concentrations of apoB and/or an apoB-containing lipoprotein and/or a component of an atherogenic lipoprotein by stimulating post-ER pre-secretory proteolysis (PERPP) using the combination of fish oils with statins, such as pravastatin, lovastatin, simvastatin, atorvastatin, fluvastatin and cerivastatin.

### SUMMARY OF THE INVENTION

The present invention is directed to a method of lipid therapy, comprising providing a subject group having a baseline triglyceride level of 200 to 499 mg/dl and being at or near its LDL-C treatment goal, and reducing the triglyceride level and the non-HDL-C level of the subject group as compared to treatment with an HMG CoA inhibitor alone, by administering to the subject group an effective amount of an HMG CoA inhibitor and a composition comprising omega-3 fatty acids.

In some embodiments, the HMG CoA inhibitor and the omega-3 fatty acids are administered as a single pharmaceutical composition as a combination product, for example, a unit dosage.

In variations of the present invention, the HMG CoA inhibitor is selected from the group consisting of pitavastatin, atorvastatin, rosuvastatin, fluvastatin, lovastatin, pravastatin and simvastatin.

In one aspect of the invention, the methods of administration and/or the combination product is used in the treatment of one or more of the following: hypertriglyceridemia, hypercholesterolemia, mixed dyslipidemia, vascular disease, atherosclerotic disease and related conditions, and/or for the prevention or reduction of cardiovascular and/or vascular events.

Other features and advantages of the present invention will become apparent to those skilled in the art upon examination of the following or upon learning by practice of the invention.

### DESCRIPTION OF THE PREFERRED EMBODIMENTS

The present invention is directed to the utilization of HMG CoA inhibitors and omega-3 fatty acids for the treatment of one or more of the following: hypertriglyceridemia, hypercholesterolemia, mixed dyslipidemia, vascular disease, atherosclerotic disease and related conditions, and/or for the prevention or reduction of cardiovascular and/or vascular events.

In some embodiments, this invention provides a method of lipid therapy, comprising providing a subject group having a baseline triglyceride level of 200 to 499 mg/dl and being at or near its LDL-C treatment goal, and reducing the triglyceride level and the non-HDL-C level of the subject group as compared to treatment with an HMG CoA inhibitor alone, by administering to the subject group an effective amount of an HMG CoA inhibitor and a composition comprising omega-3 fatty acids.

In a preferred embodiment, the administration comprises omega-3 fatty acids, preferably in the form of the Omacor® omega-3 fatty acids, and an HMG CoA inhibitor, wherein the omega-3 fatty acids are administered simultaneous to administration of the HMG CoA inhibitor, e.g., as a single fixed dosage pharmaceutical composition or as separate compositions administered at the same time.

In other preferred embodiments, the administration comprises omega-3 fatty acids and an HMG CoA inhibitor, wherein the omega-3 fatty acids are administered apart from the administration of the HMG CoA inhibitor, but in a concomitant treatment regime. For example, the HMG CoA inhibitor may be administered once daily while the omega-3 fatty acids are administered twice daily. One skilled in the art with the benefit of the present disclosure will understand that the precise dosage and schedule for the administration of the omega-3 fatty acids and the HMG CoA inhibitor will vary depending on numerous factors, such as, for example, the route of administration and the seriousness of the condition.

In some embodiments, the claimed method of administration is a first-line therapy, meaning that it is the first type of therapy given for the condition or disease. In other embodiments, the claimed method of administration is a second-line therapy, meaning that the treatment is given when initial treatment (first-line therapy, e.g., statin or omega-3 fatty acid treatment alone) does not work adequately with respect to treatment goals, or stops working adequately.

In some embodiments, the invention is suitable for primary prevention. In other embodiments, the invention is suitable for secondary prevention.

In preferred embodiments, the phrase “at or near its LDL-C treatment goal” means either at, below, or within 20% (preferably within 15%, most preferably within 10%) of the appropriate LDL-C goal set by a physician, preferably applying the standards set forth in the NCEP ATP III. The basic requirement for patients to be at or near their LDL-C goal is important because the widely accepted ATP III guidelines state that managing LDL-C should be the primary treatment objective in patients with dyslipidemia. As noted above, in the ATP III, the LDL-C goal can be calculated based on risk factors, the percentage of 10-year risk for CHD, or a combination of these elements. While the standards set forth in the ATP III are the currently preferred embodiment of the invention, this invention can also be applied using other standards of care and/or updated versions of the NCEP ATP.

In preferred embodiments, the selected subject group was receiving HMG CoA inhibitor therapy prior to the combination therapy of the HMG CoA inhibitor and the omega-3 fatty acids. Other active agents (other than omega-3 fatty acids) may also have been employed prior to the combination therapy of the HMG CoA inhibitor and the omega-3 fatty acids.

In other preferred embodiments, the method of the invention further comprises reducing at least one additional level of the subject group independently selected from the group consisting of the total cholesterol (TC) level, the apolipoprotein-B (Apo-B) level, and the very low-density lipoprotein cholesterol (VLDL-C) level, as compared to treatment with the HMG CoA inhibitor alone.

In other embodiments, the method of the invention further comprises increasing the high-density lipoprotein cholesterol (HDL-C) level, as compared to treatment with the HMG CoA inhibitor alone.

In other embodiments, the triglyceride level and the non-HDL-C level are reduced without increasing the LDL-C level, as compared to treatment with the HMG CoA inhibitor alone. In some embodiments, the triglyceride level and the non-HDL-C level are reduced without increasing LDL-C more than 1% as compared to baseline.

The phrase “compared to treatment with an HMG CoA inhibitor alone” can refer to treatment of the same subject or subject group, or treatment of a comparable subject or subject group (i.e., subject(s) within the same class with respect to a particular blood protein, lipid, or marker, such as a cholesterol or triglyceride level) in a different treatment group. The terms “reduce” and “increase” in accordance with the claimed methods are intended to mean a statistically significant reduction or increase in accordance with its general and customary meaning, i.e., a probability of chance of 5% or less (p=0.05 or less), preferably p=0.025 or less. In some embodiments of the invention, the HMG CoA inhibitor alone statistically significantly reduces or increases certain levels (such as reducing triglyceride and/or LDL-C levels or increasing HDL-C levels), and the combination therapy of the HMG CoA inhibitor and the omega-3 fatty acids further statistically significantly reduces or increases the levels.

The methods and compositions of the invention may also be used to reduce any of the following blood protein, lipid, or marker levels in a treated subject or subject group, as compared to treatment with the HMG CoA inhibitor alone: RLP-C levels, Lp-PLA2 levels and/or Apo-C3 levels.

Preferably, non-HDL-C levels may be reduced at least about 5%, preferably at least about 7%, from baseline and/or at least about 5%, preferably at least about 7%, further than treatment with the HMG CoA inhibitor alone.

Preferably, the triglyceride levels may be reduced by at least about 20%, preferably at least about 25%, as compared to baseline and/or at least about 15%, preferably at least about 20%, further than treatment with the HMG CoA inhibitor alone.

Preferably, the VLDL-C levels may be reduced by at least about 20%, preferably at least about 25%, as compared to baseline and/or at least about 15%, preferably at least about 20%, further than treatment with the HMG CoA inhibitor alone.

Preferably, the TC levels may be reduced by at least about 3%, preferably at least about 5%, as compared to baseline and/or at least about 2%, preferably at least about 3%, further than treatment with the HMG CoA inhibitor alone.

Preferably, the RLP-C levels may be reduced by at least about 20%, preferably at least about 25%, as compared to baseline and/or at least about 15%, preferably at least about 20%, further than treatment with the HMG CoA inhibitor alone.

Preferably, the Lp-PLA2 levels may be reduced by at least about 7%, preferably at least about 10%, as compared to baseline and/or at least about 5%, preferably at least about 7%, further than treatment with the HMG CoA inhibitor alone.

Preferably, the Apo-B levels may be reduced by at least about 3%, preferably at least about 4%, as compared to baseline and/or at least about 1%, preferably at least about 2%, further than treatment with the HMG CoA inhibitor alone.

Preferably, the Apo-C3 levels may be reduced by at least about 5%, preferably at least about 7%, as compared to baseline and/or at least about 8%, preferably at least about 10%, further than treatment with the HMG CoA inhibitor alone.

Preferably, the HDL-C levels may be increased by at least about 2%, preferably at least about 3%, as compared to baseline and/or at least about 3%, preferably at least about 5%, further than treatment with the HMG CoA inhibitor alone.

Preferably, the present invention also decreases the ratio of TC to HDL-C, preferably by at least about 5%, more preferably at least about 10%, as compared to baseline and/or at least about 5%, preferably at least about 10%, further than treatment with the HMG CoA inhibitor alone.

Generally, the effect of the HMG CoA inhibitor is dose dependent, i.e., the higher the dose, the greater the therapeutic effect. However, the effect of each HMG CoA inhibitor is different, and therefore the level of therapeutic effect of one HMG CoA inhibitor cannot be necessarily be directly correlated to the level of therapeutic effects of other HMG CoA inhibitors. However, those of ordinary skill in the art would understand the correct dosage to be given to a particular subject, based on experience and the seriousness of the condition.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 fatty acids (K85EE, Pronova Biocare A.S., Lysaker, Norway) and preferably comprises the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

The combination product of an HMG CoA inhibitor and omega-3 fatty acids may be administered in a capsule, a tablet, a powder that can be dispersed in a beverage, or another solid oral dosage form, a liquid, a soft gel capsule, a coated soft gel capsule (see U.S. application Ser. No. 11/716,020, hereby incorporated by reference) or other convenient dosage form such as oral liquid in a capsule, as known in the art. In some embodiments, the capsule comprises a hard gelatin. The combination product may also be contained in a liquid suitable for injection or infusion.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

Excipients include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The omega-3 fatty acids can be administered in a daily amount of from about 0.1 g to about 10 g, more preferably about 1 g to about 8 g, and most preferably from about 2 g to about 6 g. In one embodiment, the omega-3 fatty acids are administered in an amount up to 4 g/day.

The HMG CoA inhibitor may be administered in an amount more than, equal to or less than the conventional full-strength dose as a single-administered product. For example, the HMG CoA inhibitor may be administered in an amount of from 10-100%, preferably about 25-100%, most preferably about 50-80%, of the conventional full-strength dose as a single-administered product. In one embodiment of the present invention, the HMG CoA inhibitor can generally be present in an amount from about 0.5 mg to 80 mg, more preferably from about 1 mg to about 40 mg, and most preferably from about 5 mg to about 20 mg, per gram of omega-3 fatty acids. The daily dose may range from about 1 mg to about 320 mg, preferably about 2 mg to about 160 mg.

In some variations of the present invention, the combination of HMG CoA inhibitor and the omega-3 fatty acids is formulated into a single administration or unit dosage.

Pravastatin, which is known in the market as Pravachol® manufactured by Bristol-Myers Squibb, Princeton, N.J., is hydrophilic. Pravastatin is best absorbed without food, i.e., an empty stomach. The dosage of pravastatin, in the combined administration of omega-3 fatty acids is preferably from 2.5 to 80 mg, preferably 5 to 60, and more preferably from 10 to 40 mg per dosage of omega-3 fatty acids. In one variation, the combination product using pravastatin is taken at or around bedtime, e.g., 10 pm.

Lovastatin, which is marketed under the name Mevacor® by Merck, Whitehouse Station, N.J., is hydrophobic. Unlike pravastatin, lovastatin should be taken with meals and accordingly, in some embodiments, the combination product of omega-3 fatty acids and lovastatin should be taken with food. The dosage of lovastatin, in the combined administration of omega-3 fatty acids is preferably from 2.5 to 100 mg, preferably 5 to 80 mg, and more preferably from 10 to 40 mg per dosage of omega-3 fatty acids.

Simvastatin, which is marketed under the name Zocor® by Merck, Whitehouse Station, N.J., is hydrophobic. The dosage of simvastatin, in the combined administration of omega-3 fatty acids is preferably from 1 to 80 mg per day, preferably 2 to 60 mg, and more preferably from 5 to 40 mg per dosage of omega-3 fatty acids.

Atorvastatin, which is marketed under the name Lipitor® by Pfizer, New York, N.Y., is hydrophobic and is known as a synthetic statin. The dosage of atorvastatin, in the combined administration of omega-3 fatty acids is preferably from 2.5 to 100 mg, preferably 5 to 80 mg, and more preferably from 10 to 40 mg per dosage of omega-3 fatty acids.

Fluvastatin, which is marketed under the name Lescol® by Novartis, New York, N.Y., is hydrophilic and is known as a synthetic statin. The dosage of fluvastatin, in the combined administration of omega-3 fatty acids is from 5 to 160 mg, preferably 10 to 120 mg, and more preferably from 20 to 80 mg per dosage of omega-3 fatty acids.

Rosuvastatin is marketed under the name Crestor® by Astra Zeneca, Wilmington, Del. The dosage of rosuvastatin, in the combined administration of omega-3 fatty acids is from 1 to 80 mg, preferably 2 to 60 mg, and more preferably from 5 to 40 mg per dosage of omega-3 fatty acids.

Pitavastatin is currently marketed in Japan. The dosage of pitavastatin, in the combined administration of omega-3 fatty acids is from 0.25 to 20 mg, preferably 0.5 to 10 mg, and more preferably from 1 to 7.5 mg per dosage of omega-3 fatty acids.

The daily dosages of HMG CoA inhibitor and omega-3 fatty acids can be administered together in from 1 to 10 dosages, with the preferred number of dosages from 1 to 4 times a day, most preferred 1 to 2 times a day. The administration is preferably oral administration, although other forms of administration that provides a unit dosage of HMG CoA inhibitor and omega-3 fatty acids may be used.

In some embodiments, the formulations of the present invention allow for improved effectiveness of each active ingredient, with one or both administered as a conventional full-strength dose, as compared to the formulations in the prior art. In other embodiments, the formulations of the present invention may allow for reduced dosages of HMG CoA inhibitor and/or omega-3 fatty acids, as compared to the formulations in the prior art, while still maintaining or even improving upon the effectiveness of each active ingredient.

The present combination of a HMG CoA inhibitor and concentrated omega-3 fatty acids may allow for a greater effect than any expected combined or additive effect of the two drugs alone.

### EXAMPLES

Clinical study: A Randomized, Double-Blind, Placebo-Controlled Study to Assess the Efficacy and Safety of Combined Omacor® and Simvastatin Therapy in Hypertriglyceridemic Subjects

A randomized, double-blind, placebo-controlled clinical study was conducted to assess the efficacy and safety of combined treatment with Omacor® omega-3 fatty acids and simvastatin (Zocor®) in hypertriglyceridemic subjects. Patients were initially treated with 40 mg/day simvastatin for at least 8 weeks, whereupon baseline measurements were taken. Patients were eligible for enrollment and randomization if their median baseline triglyceride levels were between 200 and 499 mg/dl and their LDL-C≦10% above the NCEP ATP III goal. Initial treatment was thereafter followed by an additional 8 week treatment with either 4 g/day Omacor® omega-3 fatty acids or placebo, while continuing statin therapy, in a double-blind fashion. 243 patients completed the study.

The following Table 1 shows the results obtained for changes in various lipid and inflammatory parameters and markers.

TABLE 1

| | Omacor | Placebo | | |
|---|---|---|---|---|
| | treatment: | treatment: | | |
| | median % | median % | | |
| | change from | change from | Difference | |
| | baseline | baseline | (% median) | p-value |
| Non-HDL-C | −9.0% | −2.2% | −6.8% | <0.0001 |
| LDL-C | +0.7% | −2.8% | +3.5% | 0.0522 |
| Apo-B | −4.2% | −1.9% | −2.3% | 0.0232 |
| TG | −29.5% | −6.3% | −23.2% | <0.0001 |
| VLDL-C | −27.5% | −7.2% | −20.3% | <0.0001 |
| total C | −4.8% | −1.7% | −3.1% | 0.0013 |
| HDL-C | +3.4% | −1.2% | +4.6% | <0.0001 |
| TC/HDL | −9.6% | −0.7% | −8.9% | <0.0001 |
| RLP-C | −36.0% | −10.6% | −25.4% | <0.0001 |
| Lp-PLA2 | −12.8% | −4.7% | −8.1% | 0.0019 |
| Apo-C3 | −7.8% | +3.9% | −11.7% | 0.0002 |

The following Tables 2 and 3 show the LDL-C goal achievement experienced in the study by those on Omacor® treatment and placebo, respectively.

TABLE 2

| Omacor | | | End of Treatment | |
|---|---|---|---|---|
| treatment | Baseline | | At or below goal | Above goal |
| | At or below | 113 | 110 (97.35%) | 3 (2.65%) |
| | | (92.62%) | | |
| | Above goal | 9 (7.38%) | 3 (33.33%) | 6 (66.67%) |
| | Total | 122 (100%) | 113 (92.62%) | 9 (7.33%) |

TABLE 3

| Placebo | | | End of Treatment | |
|---|---|---|---|---|
| treatmeent | Baseline | | At or below goal | Above goal |
| | At or below | 120 | 117 (97.50%) | 3 (2.50%) |
| | goal | (90.91%) | | |
| | Above goal | 12 (9.09%) | 2 (16.67%) | 10 (83.33%) |
| | Total | 132 (100%) | 119 (90.15%) | 13 (9.85%) |

All references cited herein are hereby incorporated by reference in their entirety.

We claim:

1. A method of lipid therapy, comprising
providing a subject group having a baseline triglyceride level of 200 to 499 mg/dl and being at or near its low-density lipoprotein cholesterol (LDL-C) treatment goal, and
reducing the triglyceride level and the non-high-density lipoprotein cholesterol (non-HDL-C) level of the subject group as compared to treatment with a 3-hydroxy-3-methyl glutaryl coenzyme A (HMG CoA) inhibitor alone, by administering to the subject group an effective amount of an HMG CoA inhibitor and a composition comprising omega-3 fatty acids.

2. The method of claim 1, wherein the subject group was receiving HMG CoA inhibitor therapy prior to said method.

3. The method of claim 1, wherein the omega-3 fatty acids are administered in an amount up to 4 g/day.

4. The method of claim 1, wherein the omega-3 fatty acids are present in a concentration of at least 40% by weight as compared to the total fatty acid content of the composition.

5. The method of claim 1, wherein the omega-3 fatty acids are present in a concentration of at least 80% by weight as compared to the total fatty acid content of the composition.

6. The method of claim 1, wherein the omega-3 fatty acids comprise about 40% to about 55% by weight of EPA as compared to the total fatty acid content of the composition.

7. The method of claim 1, wherein the omega-3 fatty acids comprise about 30% to about 60% by weight of DHA as compared to the total fatty acid content of the composition.

8. The method of claim 1, wherein the omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

9. The method of claim 1, wherein the omega-3 fatty acids comprise EPA and DHA in a ratio of EPA:DHA from 2:1 to 1:2.

10. The method of claim 1, wherein the HMG CoA inhibitor is selected from the group consisting of pitavastatin, atorvastatin, rosuvastatin, fluvastatin, lovastatin, pravastatin and simvastatin.

11. The method of claim 1, further comprising reducing at least one additional level of the subject group independently selected from the group consisting of the total cholesterol (TC) level, the apolipoprotein-B (Apo-B) level, and the very low-density lipoprotein cholesterol (VLDL-C) level, as compared to treatment with the HMG CoA inhibitor alone.

12. The method of claim 1, further comprising reducing the TC level, as compared to treatment with the HMG CoA inhibitor alone.

13. The method of claim 1, further comprising reducing the Apo-B level, as compared to treatment with the HMG CoA inhibitor alone.

14. The method of claim 1, further comprising reducing the VLDL-C level, as compared to treatment with the HMG CoA inhibitor alone.

15. The method of claim 1, further comprising increasing the high-density lipoprotein cholesterol (HDL-C) level, as compared to treatment with the HMG CoA inhibitor alone.

16. The method of claim 1, wherein the triglyceride level and the non-HDL-C level are reduced without increasing LDL-C level, as compared to treatment with the HMG CoA inhibitor alone.

17. The method of claim 1, wherein the method is first-line therapy for treatment of subjects with at least one condition or disease independently selected from the group consisting of hypertriglyceridemia, hypercholesterolemia, mixed dyslipidemia, coronary heart disease (CHD), vascular disease, atherosclerotic disease and related conditions.

18. The method of claim 1, wherein the method is second-line therapy for treatment of subjects with at least one condition or disease independently selected from the group consisting of hypertriglyceridemia, hypercholesterolemia, mixed dyslipidemia, coronary heart disease (CHD), vascular disease, atherosclerotic disease and related conditions.

19. The method of claim 1, wherein the method is first-line therapy for the prevention or reduction of cardiovascular or vascular events.

20. The method of claim 1, wherein the method is second-line therapy for the prevention or reduction of cardiovascular or vascular events.
